(19) United States
(12) Patent Application Publication
(10) Pub. No.: US 2003/0186838 A1
(43) Pub. Date: Oct. 2, 2003

(54) Use of compounds having combined dopamine d2, 5-ht1a and alpha adrenoreceptor agonistic action for treating cns disorders

(76) Inventors: Roelof W. Feenstra, Weesp (NL); Johannes A.M. van der Heijden, Weesp (NL); Stephen K. Long, Weesp (NL); Gustaaf J.M. van Scharrenburg, Weesp (NL)

Correspondence Address: Finnegan Henderson Farabow, Garrett & Dunner, 1300 I Street NW, Washington, DC, 20005-3315, US

(21) Appl. No.: 10/275,813

(22) PCT Filed: May 10, 2001
(86) PCT No.: PCT/EP01/05319

(30) Foreign Application Priority Data
May 12, 2000 (EP) 002201704.4

(51) Int. Cl. A61K 31/00

(52) U.S. Cl. 514/1

(57) ABSTRACT

The invention relates to the use of compounds having combined sopamine D₂ agonistic activity, 5-HT₁A agonistic activity and a adrenoceptor agonistic activity for the treatment of CNS disorders such as Parkinson's disease.

The invention relates to the use of compounds having combined dopamine D₂-agonistic activity, 5-HT_{1A} agonistic and a adrenoceptor agonistic activity for the preparation of pharmaceutical compositions for the treatment of CNS disorders such as Parkinson's disease.

WO99/62902 describes the use of compounds having affinity for the dopamine D₂-receptor and/or the 5-HT_{1A} receptor and/or the α₁-adrenoceptor for the treatment of a large number of disorders, e.g. depression, anxiety, psychoses, obesity etc. The compounds described therein have significantly less affinity for the α₁-adrenoceptor than compounds previously described. This is said to be important as it is known in the art that α₁-receptor antagonism mediates serious side-effects such as hypertension, sedation and sexual dysfunction.

It has now been found that compounds having combined dopamine D₂-agonistic activity, serotonin 5-HT_{1A}-agonistic activity and noradrenergic α₁-adrenoceptor agonistic activity are particularly useful for the treatment of CNS disorders. Such compounds allow for a more complete treatment of Parkinson's disease without mediating the serious side-effects of compounds having the α₁-adrenoceptor antagonistic activity component.

Parkinson's disease is characterized by a slow but progressive degeneration of primarily nigrostriatal dopamine neurons. Loss of dopamine eventually results in movement disorders that are characteristic for the disorder.

It has been established that compounds with agonistic activity at dopamine D₂ receptors are beneficial in treating symptoms in Parkinson's disease. Drawback with this approach is that systems slowly desensitize and do not stop the so-called ‘wearing-off’ of pharmacotherapy. It is considered that using partial dopamine D₂ receptor agonists are equally efficacious in models for Parkinson's disease but have no or greatly reduced down-regulation of D₂ receptor sensitivity, thereby prolonging the period in which pharmacotherapy has efficacy.

Compounds that are partial dopamine D₂ agonists, i.e. with intrinsic activity between 0.3 and 0.8 (a full agonist and a full antagonist having an intrinsic activity of 1.0 and 0.0 respectively) have full efficacy in animal models for Parkinson's disease, including the induction of contralateral rotation in unilateral 6-OHDA-lesioned rats, and the MPTP-lesioned Marmoset monkeys. Interestingly, depending on intrinsic activity compounds may act as partial agonists in partially denervated systems such as reserpinized rats. Moreover, all compounds are active in classical dopamine agonist models such as climbing behavior and locomotion in rats and mice.

Intrinsic activity lower than 0.3 produces truly partial efficacy in animal models. This is parallelled by an increase in dosage necessary to achieve behavioral responses.

Interfering with central dopamine systems is also likely to interupt reward-seeking behavior which is associated with psychological and physical dependence (addiction) as well as withdrawal, and impulsive disorders like Gilles de la Tourette syndrome. Furthermore, there is evidence that dopaminergic agonists may be effective also in the treatment of anxiety symptoms.

Underneath the neurological symptoms related to Parkinson's disease, more than half of the patients also suffer from mood disorders, which affect the quality of life in many cases immensely. This may be attributed at least in part to the neurodegeneration of the serotonergic raphe nuclei and the noradrenalin-producing cells in the locus coeruleus, although this degeneration lags behind that of the dopaminergic substantia nigra.

Thus by combining partial D₂ receptor agonism with 5-HT_{1A}, and α-adrenoceptor agonism, treatment of the primary Parkinson's disease symtoms, like bradykynesia, resting tremor, stiffness and rigidity, and in addition secondary symptoms, like depression, panic, generalized anxiety and dementia, will be possible.

Compounds having this unique combination of pharmacological activities are indeed not only active in animal models for Parkinson's disease but are also active in animal models predictive for anxiolytic behavior, such as the adult ultrasonic vocalization and stress-induced hyperthermia, as well as in animal tests that are predictive for anti-depressant activity, such as the forced swim test.

In conclusion, such compounds are extremely potent, display partial agonism at dopamine D₂ receptors for prime treatment of movement disorders such as Parkinson's disease, and include agonism at serotonin 5-HT_{1A} and noradrenergic α-adrenoceptors to treat mood disorders and dementia, and may also be effective in treating dependence (addiction).

The combination of three desired types of activity into one molecule has a number of advantages in view of combining three different active components in a composition:

1. constant ratio of all activities due to one kinetic behaviour

2. less burden for the body of the patient with chemical compounds

3. less possibilities for undesired side-effects

4. no interaction between active components.

The invention is illustrated by but is not restricted to the use of the group of compounds having formula (I)

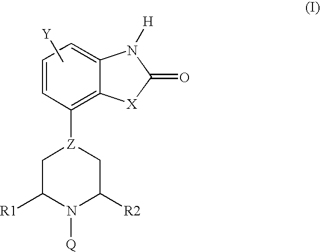

wherein

Y is hydrogen, halogen, alkyl (1-3C), or CN, CF₃, OCF₃, SCF₃, alkoxy(1-3C), amino or mono- or dialkyl(1-3C) substituted amino or hydroxy,

X is O, S, SO or SO₂,

-Z represents —C, ═C or —N,

R₁ and R₂ independently represent hydrogen or alkyl (1-3C),

Q is benzyl or 2-, 3- or 4-pyridylmethyl, wich groups may be substited with one or more substituents from the group halogen, nitro, cyano, amino, mono- or di (1-3C)alkylamino, (1-3C) alkoxy, CF₃, OCF₃, SCF₃, (1-4C)-alkyl, (1-3C)alkylsulfonyl or hydroxy, and salts and prodrugs thereof.

The compounds are their acid addition salts can be brought into forms suitable for administration by means of suitable processes using auxiliary substances such as liquid and solid carrier materials.

Preferrably the compounds of the invention have a high oral bioavailibility (F) which is at least higher than 30% and even more preferred higher than 50%.

The compounds having formula (I) can be obtained as follows:

Method A

Compounds having formula (I) wherein -Z represents —N or —C can be obtained by reacting the corresponding compound wherein Q is hydrogen with a compound Q-Hal, wherein Q has the above meanings and Hal is halogen, preferably bromine. This reaction can be carried out in a solvent such as acetonitrile in the presence of a base, for example ethyl-diisopropylamine or triethylamine.

The starting compounds wherein Q is hydrogen and -Z is —N are known or can be obtained as described in EP 0189612. Starting compounds wherein Q is hydrogen and -Z is —C can be obtained as described below in schema A.i (compound III-H).

Method B

The compounds B1, i.e compounds having formula (I) wherein -Z represents ═C can be obtained according to the method indicated in the following scheme A.i:

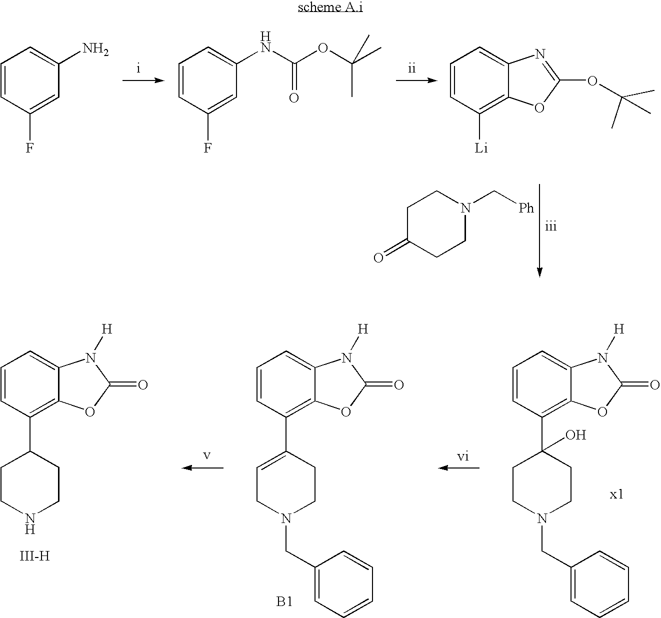

The starting compound for step (ii) can be obtained according to the procedure described in J. Org. Chem. 45,(1980), 4789, and step (ii) itself can be carried out as described in J. Org. Chem, 47, (1982), 2804.

Step (iii) is carried out in a manner known for this type of chemical reactions.

The preparation of the compounds having formula (I) will be illustrated in the following Examples:

### EXAMPLE 1

General Procedure for method A:

a) To 1 mmol of halide Q-Hal, 0.8 mmol of I—H (-Z=—N) dissolved in 7.5 ml of CH₃CN was added. Subsequently 0.43 ml (2.5 mmol) of (i-Pr)₂NEt was added and the resulting mixture was stirred for 3 hrs at 85° C. After the reaction mixture had reached roomtemperature, 7.5 ml of dichloromethane were added, the resulting solution was put on top of a solid phase extraction column (Varian 5 g type Si) and the fraction containing the desired product was subsequently put on top of a solid phase extraction column (Varian 5 g 0.8 meq./g type Strong Cationic Exchange (SCX), conditioned on MeOH, then CH₂Cl₂)) after which the column was washed 2 times with MeOH. Then, the latter column, was washed with 0.1 M NH₃/MeOH and elution was performed with 1.0 M NH₃/MeOH. The eluate was concentrated in vacuo removing solvent and the rest of (i-Pr)₂NEt, yielding the expected product.

It is also possible to perform the purification with standard chromatographic procedures. In a single case (i.e. Al), the solvent used was dimethylformamide (DMF), see below.

b) 10.2 g (40 mmol) of I-H.HCl were suspended in 150 ml of DMF, to the stirred resulting mixture 21 ml (120 mmol) of (i-Pr)₂NEt were added. During a period of 10 minutes a solution of 7.0 g (41 mmol) of benzylbromide in 25 ml of DMF was added at room temperature, the process is slightly exothermic (5-10° C.). Stirring was continued 3 hrs at room temperature after which the reaction mixture was poured on to 700 ml of water. Subsequently extraction was performed with 3×250 ml of ethylacetate, the combined organic fractions washed with 2×150 ml of water and dried with MgSO₄. Removal of the drying agent by filtration and of the solvent in vacuo yielded 10.5 g of raw product. The latter was purified by flash column chromatography (SiO₂, eluent CH₂Cl₂/MeOH 98/2), yielding 8.5 g (69%) of pure product Al as a free base, m.p.: 189-190° C.

The compounds A2 to A46 as indicated in table A have been prepared analogously to procedure a) of method A.

TABLE A

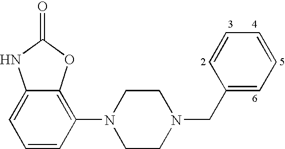

| | | | | | position(s) | | | | |
|---|---|---|---|---|---|---|---|---|---|
| com- | | | melting | | substitution(s) | | | | |
| pound | Hal | salt | point ° C. | | 2 | 3 | 4 | 5 | 6 |
| A1 | Br | fb | 189-90 | | | | | | |
| A2 | Br | fb | 220-22 | d | Br | | | | |
| A3 | Br | fb | 170-2 | d | F | F | F | F | F |
| A4 | Br | fb | 220-2 | d | | | CN | | |
| A5 | Br | fb | 130-2 | d | | OMe | | | |
| A6 | Br | fb | 223-5 | d | | | SO₂Me | | |
| A7 | Br | fb | 235-7 | d | Cl | | | | Cl |
| A8 | Br | fb | 190-2 | d | F | Me | | | |
| A9 | Br | fb | 200-2 | d | F | F | | | |
| A10 | Br | fb | 122-4 | d | | SCF₃ | | | |
| A11 | Br | fb | >250 | d | Cl | | | Cl | |
| A12 | Br | fb | 160-70 | d | Me | | | | |
| A13 | Cl | fb | 165-7 | d | | | OMe | | |
| A14 | Br | fb | 177-9 | | | F | F | | |
| A15 | Br | fb | 150-2 | | | | OCF₃ | | |
| A16 | Br | fb | 146-8 | | | Br | | | |
| A17 | Br | fb | 193-5 | | | Br | OMe | | |
| A18 | Br | fb | 170-1 | | | F | F | F | |
| A19 | Br | fb | 195-7 | | F | | F | | |
| A20 | Br | fb | 171-3 | | OCF₃ | | | | |
| A21 | Br | fb | 191-6 | d | | Cl | Cl | | |
| A22 | Br | fb | 183-6 | | | Me | | | |
| A23 | Br | fb | 132-4 | | | | CF₃ | | |
| A24 | Br | fb | 194-206 | d | | F | | F | |
| A25 | Br | fb | 124-7 | | | CF₃ | | | |
| A26 | Br | fb | 184-6 | | | | tBut | | |
| A27 | Br | fb | 216-8 | d | Cl | | | | |
| A28 | Br | fb | 115-20 | | | CF₃ | F | | |
| A29 | Br | fb | 175-8 | | CF₃ | | | | |
| A30 | Br | fb | 186-8 | | Cl | | | CF₃ | |
| A31 | Br | fb | 197-200 | | F | | F | | F |
| A32 | Br | fb | 159-63 | | | | Br | | |
| A33 | Cl | fb | 152-8 | d | | Me | Me | | |
| A34 | Br | fb | 178-83 | | F | | | | |
| A35 | Br | fb | 215-9 | | CN | | | | |
| A36 | Br | fb | 198-200 | | | Me | | Me | |
| A37 | Br | fb | 190-5 | | | | Me | | |
| A38 | Br | fb | 166-76 | | | CN | | | |
| A39 | Br | fb | 188-90 | | CF₃ | | F | | |
| A40 | Br | fb | 210-4 | | Cl | | F | | |
| A41 | Br | fb | 180-6 | | | | F | | |
| A42 | Br | fb | 159-63 | | | F | | | |
| A43 | Br | fb | 178-80 | | F | Cl | | | |

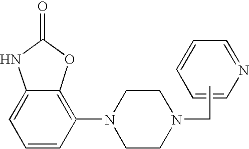

| | | | melting | | |
|---|---|---|---|---|---|
| compound | Hal | salt | point ° C. | | Q |
| A44 | Cl | fb | 188-90 | d | 2-pyridylmethyl |
| A45 | Cl | fb | 175-9 | | 3-pyridylmethyl |
| A46 | Cl | fb | 230-5 | d | 4-pyridylmethyl |

d = decomposition
fb = free base

### EXAMPLE 2

Step ii and iii (Scheme A.i):

Under an inert atmosphere, 16.5 g (78.2 mmol) of N-(tert.butyloxycarbonyl)-meta-fluoroaniline were dissolved in 230 ml of dry tetrahydrofuran (THF) after which the solution was cooled to −75° C. (dry ice, acetone). While stirring, a solution of tert.butyl-lithium in heptane (ca. 156 mmol, 2 molequivalents) was added slowly after which the reaction mixture was stirred for 0.5 hrs at −70° C., and subsequently for an additional 2 hrs at −25° C. Again the reaction mixture was brought to −75° C. and a solution of 14.4 ml N-benzylpiperidone (78 mmol, 1 molequivalent) in 25 ml of dry THF. The reaction mixture was allowed to reach room temperature and stirred for an additional 16 hrs. Subsequently, 250 ml of 2M HCl was carefully added, the resulting mixture was extracted with EtOAc (3×). The water layer was, while stirring, poured on to 84 g of NaHCO₃ after which the waterlayer was again extracted with EtOAc. The resulting organic layer was dried on Na₂SO₄. After removal of the drying agent by filtration and of the solvent by evaporation in vacuo, 15 g of a dark yellow oil was isolated. Column chromatography (SiO₂, eluent: CH₂Cl₂MeOH 911) yielded 7.5 g (ca. 30%) of a light yellow foam. While stirring, 1 g of the foam was triturated with di-ethyl ether and a small volume of EtOAc. After 50 hrs the solid material was filtered and washed with with di-ethyl ether/hexane to yield 0.5 g of a nearly white solid x1, mp 125-8 DC.

Step iv (Scheme A.i):

While stirring, 6.3 g (19.4 mmol) of x1 (scheme A.i.) was dissolved in 250 ml of dioxane after which 150 ml of concentrated HCl was added, the resulting mixture was refluxed for 1.5 hrs. The reaction mixture was allowed to reach room temperature after which it was poured on to 140 g of NaHCO₃, subsequently about 250 ml of EtOAc were added and an amount of water enough to solve all of the solid material, the pH was >7. The layers were separated and the waterlayer was extracted with EtOAc (2×). The combined organic fractions (3), were dried on Na₂SO₄. After removal of the drying agent by filtration and of the solvent by concentration in vacuo, 8 g of a dark yellow oil was isolated which solidified on standing. Column chromatography (SiO₂, eluent: EtOAc) yielded 4.56 g (ca. 30%) of a nearly white product. The latter was suspended in hexane and stirred for 20 hrs. Filtration and drying of the residue yielded 3.5 g (59%) of a white solid BI as a free base, mp ca. 153° C.

### EXAMPLE 3

Preparation of Intermediate III-H of Scheme A.i.

Step v (Scheme A.i):

2.71 g (8.9 mmol) of BI of scheme A.i. were dissolved in 250 ml of absolute EtOH. To the latter solution 0.6 g of 20% Pd(OH)₂ on carbon was added after which the reaction mixture was subjected to hydrogenation for 18 hrs at roomtemperature. Subsequently the reaction mixture was filtered (hyflo supercel) and the residu (hyflo) washed with methanol/triethylamine 97/3. The filtrate was concentrated in vacuo yielding 1.87 g of a nearly white solid which was suspended in EtOAc and stirred for 20 hrs. Filtration of the solid and subsequently drying afforded 1.56 g (81%) of the intermediate III-H (scheme A.i.).

1. Use of a compound having combined dopamine D₂-agonistic activity, 5-HT_{1A} agonistic and α adrenoceptor agonistic activity for the preparation of pharmaceutical compositions for the treatment of CNS disorders.

2. Use as claimed in claim 1, characterized in that said compound is used for the treatment of Parkinson's disease.

3. Use as claimed in claim 1-2, characterised in that a compound having pKi-values of more than 7.5 in the receptor binding assays for D₂-receptors, 5-HT_{1A} receptors and α₁ adrenoceptor is used.

4. Use as claimed in claims 1-3, characterised in that the dopamine D₂ activity of said compound is a partial agonistic activity with an intrinsic activity between 0.3 and 0.8

5. Use as claimed in claims 1-4, characterised in that the 5-HT_{1A} activity of said compound is a full agonistic activity.

6. Use as claimed in claims 1-4, characterised in that the ax adrenoceptor agonistic activity of said compound is a full agonistic activity.

7. Use as claimed in claims 1-6, characterized in that said compound has additionally dopamine D₁ agonistic activity of said compound is a full agonistic activity.

8. Use as claimed in claims 1-7, characterized in that said compound has additionally dopamine D₄ activity with a pki-value of more than 7.5 in the binding assay for D₄-receptors
